(19) United States
(12) Patent Application Publication
(10) Pub. No.: US 2023/0137188 A1
(43) Pub. Date: May 4, 2023

(54) HYPERPOLARISATION DEVICE, SYSTEM AND PROCESS

(71) Applicant: MASTER DYNAMIC LIMITED, Hong Kong (CN)

(72) Inventors: Yau Chuen YIU, Hong Kong (CN); Juan CHENG, Hong Kong (CN); Koon Chung HUI, Hong Kong (CN)

(73) Assignee: MASTER DYNAMIC LIMITED, Hong Kong (CN)

(21) Appl. No.: 17/802,470

(22) PCT Filed: Feb. 26, 2021
(86) PCT No.: PCT/CN2021/078232

§ 371 Date: Aug. 25, 2022

(30) Foreign Application Priority Data
Feb. 26, 2020 (HK) 32020003350.6

(51) Int. Cl. G01R 33/56; G01R 33/28

(52) U.S. Cl. CPC G01R 33/5601; G01R 33/282

(57) ABSTRACT

A device(100a(i), 100a(ii), 100a(iii), 100a(iv), 200 for cis-tracing polarization of ¹²C isotope-based magnetic resource imaging contrast agents, comprising one or more diamond material structures(120(i), 120(ii), 120(iii), 120(iv), 210, 220) and one or more channels(105a(i), 105a (ii), 105a (iii), 105a (iv), 205) provided adjacent to the diamond material structures 120(i), 120(ii), 120(iii), 120(iv), 210, 220); the diamond material structures 120(i), 120(ii), 120(iii), 120(iv), 210, 220) provide a source of negatively charged nitrogen vacancy celles for polarization of a ¹³C iso-type-based magnetic measure imaging contrast agesi disguised in one or more channels(105a (i), 105a (ii), 105a (iii), 105a (iv), 205) and cis diamond material structure(120(i), 120(ii), 120(iii), 120(iv), 210, 220) provides a light guide for light for excitation of nitrogen vacancy celles for polarization of ¹³C isotope-based magnetic resource imaging contrast agent.

### TECHNICAL FIELD

The present invention relates to a device, process and system for hyperpolarizing ¹³C isotope-based Magnetic resonance imaging contrast agents for subsequent magnetic resonance imaging (MRI) applications.

### BACKGROUND OF THE INVENTION

Magnetic resonance imaging (MRI) has been widely used in the medical discipline for obtaining the three-dimensional structural information from a human body.

By obtaining a three-dimensional image, medical practitioners are able to see through the organs of a patient, and determine if there are any structural abnormalities within the body of the patient.

One such abnormality is the presence of tumour tissue. Traditional MRI techniques detect 1H nuclei inside human bodies, such that the water and fat distribution can be seen. Since no ionizing radiation is involved, it is considered to be a safer investigation method than X-ray imaging techniques.

However, detecting 1H nuclei alone cannot always distinguish normal tissue and cancerous tissue and as such the techniques can be considered to be less applicable than X-ray computed tomography (CT) and positron emission tomography (PET).

Therefore, in order to enhance the contrast between normal and cancerous tissue, contrast agents are needed to be introduced into the body. These MRI contrast agents typically contain gadolinium, which, however, has certain toxicity towards the kidneys and the nervous system of a patient.

Patients with renal diseases are considered susceptible to kidney failure after injection of gadolinium-based contrast agents into the body. Moreover, gadolinium can remain in human body for a prolonged period time after MRI scanning, which also increases the risk of patient safety related issues.

Apart from gadolinium-based contrast agents, there has been some research on ¹³C nuclei based MRI imaging to distinguish normal and cancerous tissues. Carbon, as is known, is the building block of all organic compounds.

Since ¹³C nuclei are stable, there is considered no harm in using ¹³C for MRI imaging in living organisms. However, the natural abundance of ¹³C nuclei in carbon is only 1.1%, which is much smaller than the natural abundance of 99.98% of 1H nuclei in hydrogen. Moreover, ¹³C signal in MRI is much weaker than 1H.

These two factors together can be considered to make MRI by ¹³C very difficult practically. Nevertheless, there have been technologies for enriching ¹³C abundance in biomolecules. Therefore, ¹³C enhanced compounds with high purities can be obtained commercially.

Regarding the low signal of ¹³C in comparison to 1H, there are also techniques for enhancement in the art. At room temperature, the nuclear spin alignment of ¹³C within a magnetic field is little under thermal equilibrium.

### OBJECT OF THE INVENTION

It is an object of the present invention to provide a device, process and system for hyperpolarizing ¹³C isotope-based Magnetic resonance imaging contrast agents for subsequent magnetic resonance imaging (MRI) applications, which overcomes or at least partly ameliorates at some deficiencies as associated with the prior art.

### SUMMARY OF THE INVENTION

In a first aspect, the present invention provides a device for enhancing polarization of ¹³C isotope-based magnetic resonance imaging contrast agents, said device comprising:
one or more diamond material structure and one or more channels provided adjacent to the diamond material structure;
wherein said diamond material structure provides a source of negatively charged nitrogen vacancy (NV-) centres for polarization of a ¹³C isotope-based magnetic resonance imaging contrast agent disposed in said one or more channels; and;
wherein said diamond material structure provides a light guide for light for excitation of nitrogen vacancy (NV-) centres for polarization of said ¹³C isotope-based magnetic resonance imaging contrast agent.

The device may include a plurality of plurality of columns defining said channels therebetween, wherein said columns are formed from said diamond material structure;

The diamond material structure may be formed from synthetic diamond.

The diamond material structure may be formed from CVD (chemical vapor deposition crystal formation) diamond.

The diamond material structure may be formed from HPHT (high-pressure high-temperature) diamond.

The light may be applied by an optical laser. The light may be provided by a pulse laser. The light is provided by a continuous laser. The light may be monochromatic.

The ¹³C isotope-based magnetic resonance imaging contrast agents may pyruvate.

In a second aspect, the present invention provides a system for enhancing polarization of ¹³C isotope-based magnetic resonance imaging contrast agents, said system comprising:
a device according to the first aspect
a light source for optical pumping and providing excitation to the electron spins of the nitrogen vacancy (NV-) centres;
a radio frequency transmitter for providing a radio frequency signal;
a microwave transmitter for providing a microwave field, such that the NV centres are polarized and such that the electron spins of the NV centres are transferred to ¹³C atoms upon the Rabi frequency of the NV centres matching the Larmor frequency of ¹³C.
a tuneable electromagnet for providing a magnetic field, such that the nitrogen vacancy (NV-) centres electron spin active states of the nitrogen vacancy (NV-) centres are sufficiently separated, in order to provide unified electron spins states within the diamond under the presence of a microwave field.

The radio frequency signal may be a static radio frequency wave. The radio frequency signal is a pulsed signal. The microwave may be a continuous wave. The microwave may be a pulsed signal.

The electromagnet may be a tuneable magnet.

In a third aspect, the present invention provides a process for enhancing polarization of ¹³C isotope-based magnetic resonance imaging contrast agents for subsequent MRI imaging, said process including the steps of:
(i) providing a device according to the first aspect;
(ii) introducing a ¹³C isotope-based magnetic resonance imaging contrast agent with the channels of said device;
(iii) polarizing said ¹³C isotope-based magnetic resonance imaging contrast agent.

In a fourth aspect, the present invention provides a system for cleaning and disinfecting the device for enhancing polarization of ¹³C isotope-based magnetic resonance imaging contrast agents of the first aspect, said system comprising:
(i) a disinfection chamber, for cleaning and disinfecting said one or more devices simultaneously;
(ii) a disposal chamber, wherein any waste retained on said one or more devices are disposed from the system, and
(iii) a drying chamber for drying the cleaned one or more said devices.

The disinfection chamber may include an ultraviolet light source for disinfection of said one or more devices. The ultraviolet light source may be in the wavelength of 200 nm to 400 nm. The ultraviolet light source may be provided by LED or laser.

The disinfection chamber may have a reservoir for storing organic liquid, and any kinds of water, which allows said one or more devices to be cleaned therein.

The drying chamber may utilise furnace or electromagnetic wave as heat source for the drying purpose. The drying chamber may include an ultraviolet light source for disinfection of said one or more devices. The ultraviolet light source may be in the wavelength of 200 nm to 400 nm. The ultraviolet light source is provided by LED or laser.

In a fifth aspect, the present invention provides a process for enhancing the hyperpolarizing effect of the ¹³C -isotope based contrast agent provided by the device of the first aspect, said process including the steps of:
(i) treating said diamond material structure such that the diamond material is plamonic-based or photonic-based;
(ii) illuminating a light source to said diamond material structure for optical pumping and providing excitation to the electron spins of the nitrogen vacancy (NV-) centres, and as such to provide surface plasmons on the surface of said diamond material structure; and
(iii) providing electromagnetic perturbation to said device.

The diamond material may be treated by forming a metallic coating on the surface of the diamond material structure. The diamond material may be treated by coupling the diamond material with metallic micro or nanostructures. The diamond material may be treated by forming a dieletric coating on the surface of the diamond material structure. The diamond material may be is treated by coupling the diamond material structure with dieletric micro or nanostructures.

The electromagnetic perturbation may be provided by any one of microwave source, radio frequency wave source, or the like.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed.

FIG. 1a (i) - FIG. 1a (iv) show schematic exemplary examples of a device according to the present invention;

FIG. 1b shows a schematic representation of a system in accordance with the present invention;

FIG. 2 shows a schematic representation of an embodiment of a hyperpolarization device according to the present invention;

FIG. 3 shows a schematic representation of an embodiment of a hyperpolarization casing according to the present invention;

FIG. 4 shows a schematic representation of the formation of surface plasmons on the surface of the plasmonic-based or photonic based diamond;

FIG. 5a shows an energy diagram of a nitrogen vacancy of a diamond upon excitation by focused light, without the presence of surface plasmons on the diamond surface;

FIG. 5b shows an energy diagram of a nitrogen vacancy of a diamond upon excitation by focused light, with the presence of surface plasmons on the diamond surface; and

FIG. 6 shows a schematic representation of an embodiment of the disinfection system of one or more hyperpolarisation devices according to the present invention.

### DETAILED DESCRIPTION OF THE DRAWINGS

The present inventors have identified shortcomings of the problems with the prior art, and have provided a system which is more consistent and reliable, and overcomes the problems of the prior art.

### Present Invention

The present invention relates to hyperpolarization of enhancing the ¹³C isotope-based Magnetic resonance imaging (MRI) contrast agents. The present inventors have provided a process, device and system to increase the efficiency of optical hyperpolarization ¹³C isotope-based Magnetic resonance imaging contrast agents.

The present inventors have developed a process, device and system for enhancing the ¹³C isotope-based Magnetic resonance imaging (MRI) contrast agents using optically pumping, which can be performed at room temperature.

### Invention Background Theory

It is known that cancer cells exhibit a unique metabolic fingerprint that provides a means to differentiate them from benign tissues. In order to investigate the cancer cells, Magnetic resonance imaging is one of the tools.

In particular, ¹³C Magnetic resonance imaging (MRI) is attractive for metabolic imaging because carbon serves as backbone of nearly all organic molecules, thus allowing the investigation in the area of cancer metabolism.

However, in practice, the signal from a ¹³C labelled tracer has been considered too weak for in vivo imaging due to the very low natural abundance of the ¹³C isotope.

In order to improve the MRI signal of ¹³C nuclei, detection probes can be synthetically enriched to increase the concentration of the ¹³C label in a molecule. MRI signal can be further enhanced dramatically by the process of hyperpolarization.

In order to enhance the ¹³C signal, the ratio of aligned nuclear spin under magnetic field is needed to be greatly increased beyond thermal equilibrium. This phenomenon is called hyperpolarization within the art.

Dynamic nuclear polarization (DNP) is a method which can hyperpolarize ¹³C so that ¹³C signal can be enhanced by 10,000-fold compared to thermal equilibrium in room temperature. This makes use of compounds with radicals to provide lone pair electrons, whose aligned spins can polarize the nuclear spins of ¹³C. By adding radicals into ¹³C compounds at around 1 K in a magnetic field of 4.6 T to 5 T for 30 min to 90 min, the ¹³C nuclear spin can be hyperpolarized.

As the radicals used in DNP have certain toxicity to human cells and the DNP process has to be done in cryo-environment, there have been proposed other methods developing for the hyperpolarization of ¹³C.

The principle of hyperpolarization is the high spin polarization of a paramagnetic radical can be transferred to the ¹³C nucleus on another molecule under resonant microwave irradiation.

However, conventional methods generally involve the conditions of low temperature (~<=K) and high magnetic field (>=3 T) to first generate the electron polarization. It is reported the hyperpolarization signal of ¹³C nuclei can be increased with a factor of 720 against the thermal signal at 7 T and retained for multiple-minute long period via optical hyperpolarization.

Diamonds contain Nitrogen Vacancy (NV) centres with one negative charge captured from the surroundings. The diamond NV- centres are paramagnetic with spin S = 1 with a large zero field splitting, with D = 2.87 GHz, wherein D is the energy difference between electron spin state of zero-field splitting of NV center, the energy range is in microwave band.

Laser light can be used for optical pumping, providing excitation, to the electron spins of NV centres. The electron spins of the NC centres can then be transferred to ¹³C atoms when the Rabi frequency of the NV centres match the Larmor frequency of¹³C.

### Present Invention Details

In accordance with the present invention, a process, device and system has been proposed and developed to enhance the efficiency of optical pumping for hyperpolarizing ¹³C isotope-based Magnetic resonance imaging contrast agents for subsequent magnetic resonance imaging (MRI) applications.

The present invention achieves such enhanced efficiency of optical pumping for hyperpolarizing ¹³C isotope-based Magnetic resonance imaging contrast agents for subsequent magnetic resonance imaging (MRI) applications.

The manner in which the present inventors have overcome the deficiencies of the prior art and provided a solution which can enhance the efficiency of optical pumping, is by providing a hyperpolarization device which include a plurality of channels in which ¹³C isotope-based Magnetic resonance imaging contrast agents, either as a solution or in particulate form, may be introduced therein.

Advantageously, in order achieve the objectives of the invention, the material surrounding or adjacent the channels, is formed from diamond, preferably a synthetic diamond, which is also known as laboratory-grown diamond, or laboratory-created diamond.

Such diamonds are produced by a controlled process, as contrasted with natural diamonds which are created by geological processes, or imitation diamond made of non-diamond material that appears similar to diamond.

Synthetic diamonds are also widely known as HPHT (high-pressure high-temperature) diamond or CVD diamond (chemical vapor deposition crystal formation), as being formed from such methods.

As required for the hyperpolarization of ¹³C isotope-based Magnetic resonance imaging (MRI) contrast agents, as the hyperpolarization device of the present invention includes a diamond material structure that is formed from diamond material, this diamond material of the diamond material structure provides the source of negatively charged nitrogen vacancy (NV-) for hyperpolarization of the ¹³C isotope-based Magnetic resonance imaging (MRI) contrast agents, for use in subsequent MRI imaging of a patient.

As such, the hyperpolarization device provides for enhanced hyperpolarization of the ¹³C isotope-based Magnetic resonance imaging (MRI) contrast agents, as it acts as:
(i) the diamond material structure acts as a light guide for light as is required for hyperpolarization, and further into adjacent MRI contrast agents, thus providing greater penetration and increased hyperpolarization of MRI contrast agents, and
(ii) the diamond material structure provides the source of negatively charged nitrogen vacancy (NV-) as is required for hyperpolarization of the MRI contrast agents in accordance with the present invention.

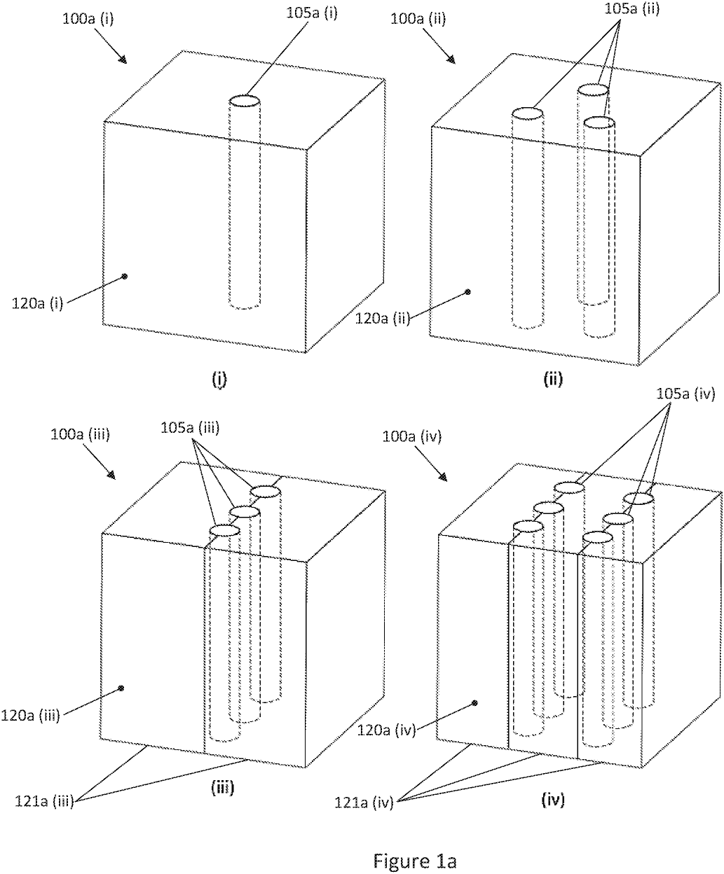

Referring now to FIG. 1a (i) - FIG. 1a a(iv), there are shown schematic exemplary examples of a device 100a(i), 100a(ii), 100a(iii) and 100a(iv) for enhancing polarization of ¹³C isotope-based magnetic resonance imaging contrast agents, according to the present invention.

The device 100a(i), 100a(ii), 100a(iii) and 100a(iv) includes one or more diamond material structure 120a(i), 120a(ii), 120a(iii) and 120a(iv) and one or more channels 105a(i), 105a(ii), 105a(iii) and 105a(iv) provided adjacent to the diamond material structure 120a(i), 120a(ii), 120a(iii) and 120a(iv).

The diamond material structure 120a(i), 120a(ii), 120a(iii) and 120a(iv) provides a source of negatively charged nitrogen vacancy (NV-) centres for polarization of a ¹³C isotope-based magnetic resonance imaging contrast agent disposed in said one or more channels; and;

Further, the diamond material structure 120a(i), 120a(ii), 120a(iii) and 120a(iv) provides a light guide for light for excitation of nitrogen vacancy (NV-) centres for polarization of said 13C isotope-based magnetic resonance imaging contrast agent.

As will be seen in FIG. 1a (i) and FIG. 1a (ii), the diamond material structure 120a(i), 120a(ii), is unitary in structure, with channels 105a(i), 105a(ii) extending therethrough. As will be understood, there may be a plurality of channels or a single channel, in accordance with the present invention.

Now as will be seen in FIG. 1a (iii) and FIG. 1a (iv), the diamond material structure 120a(iii), 120a(iv) is not a unitary structure and is provided by separate components 121 a(iii),121 a(iv).

It will be understood that not all of the separate components 121a(iii),121a(iv) necessarily formed from a diamond material, however as long as sufficient diamond material is provided adjacent to a channel, the hyperpolarization process according to the present invention may be effected.

As will be understood, the channels 105a(iii) and 105a(iv) in the devices 100a(iii) and 100a(iv) a formed when the separate components 121 a(iii),121 a(iv). are joined together.

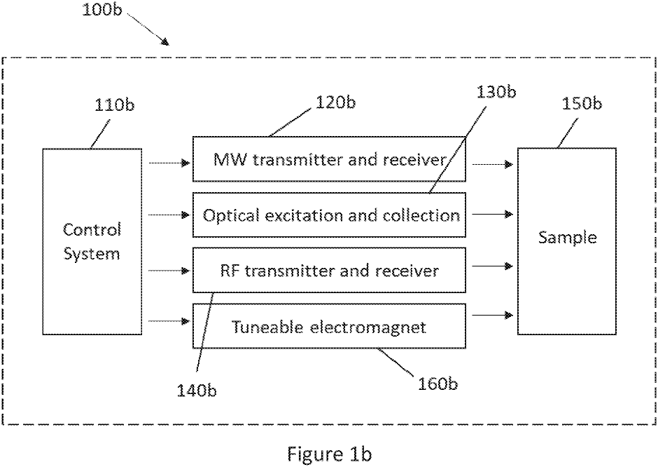

As will also be understood, the channels 105a(i), 105a(ii), 105a(iii) and 105a(iv) suitably sized so as to provide for the hyperpolarization process as will be described further in detail in particular, in reference to FIG. 2 as follows.

Further, as will be understood, the channels 105a(i), 105a(ii), 105a(iii) and 105a(iv) need not necessarily be circular, and may be of any geometry and the present embodiments impart no geometric or size constraint in respect of the channels. 105a(i), 105a(ii), 105a(iii) and 105a(iv). Furthermore, although the channels 105a(i), 105a(ii), 105a(iii) and 105a(iv) depicted in the exemplary embodiments extend fully through the diamond material structure 120a(i), 120a(ii), 120a(iii) and 120a(iv), this need not necessarily be the case in all implementations of the invention, and again, the present environments are exemplary only and do not provide any limitation on the structure of the device 100a(i), 100a(ii), 100a(iii) and 100a(iv).

Further and advantageously, in embodiments of the invention, the hyperpolarization device 100a(i), 100a(ii), 100a(iii) and 100a(iv) can be incorporated into a hyperpolarization system, with requisite microwave generator, a magnetic field generator, and a laser light source for delivery of light of a requisite predetermined wavelength.

Referring to FIG. 1b, there is shown a schematic representation of a system 100b in accordance with the present invention which may implement the device of the present invention.

The present invention provides a system, device and process for enhancing ¹³C isotope MRI imaging signal via optically hyperpolarization at room temperature.

The system 100b can be placed in a same room with MRI (Magnetic Resonance Imagining) machine or in a separated room.

FIG. 1b shows a schematic representation of the system 100b which includes a controller 110b for controlling the operation of an optical excitation and collection device 130b, a microwave (MW) transmitter and receiver 120b for providing a microwave field, as well as radio frequency (RF) transmitter and receiver 140b for providing a radio frequency, and tuneable electromagnet 160b for providing tuneable magnetic field.

The device 150b can be exposed to microwave, radio frequency, magnetic field and a light source.

The presence of magnetic fields allows the degenerated electron spin states splitting to non-degenerated electron spin sublevels in the order of microwave energy scale.

Degenerated electron spin states can be split and separated by magnetic field, which is known as the Zeeman effect, the sublevels are separated by very small amount of energy in the microwave region. Upon the illumination of a light energy, the sample can be excited from ground electronic and spin states to excited electronic and spin states. The sample will then decay back to ground state by releasing energy in form of light or heat.

During the excitation and decay processes, the electron spin states of the sample could be switched between fluorescence-active and fluorescence-inactive electron spin states. Microwave will be absorbed by the sample when there is any allowed transition between any two fluorescence-active and fluorescence-inactive electron spin states.

As such, the magnetic field splits and separate the degenerated electron spin states of the sample due to Zeeman effect, and thus providing unified electron spins states within the sample under the presence of a microwave field.

Without the presence of a magnetic field, the electron spins of NV centres within the sample cannot be unified and thus would be cancelled out.

Any kind microwave transmitter 120b can be used, and as such to provide pulsed or continuous microwave. The pulsed or continuous microwave can be applied on the device 130b uniformly or nonuniformly.

Any kind radio frequency transmitter 120b can be used, and as such to provide pulsed or continuous radio frequency. The pulsed or continuous radio frequency can be applied on the device 130b uniformly or nonuniformly.

Any kind electromagnet 160b can be used, and as such to provide pulsed or continuous magnetic field on sample. The pulsed or continuous magnetic field can be applied on the sample uniformly or nonuniformly.

Light can be provided by a laser light source 130b for example. The light may be pulsed light or continuous light. Preferably monochromatic light is used. Although the light source is preferably a laser light source, other light sources may be utilized in alternate configurations and embodiments. A magnetic field and a microwave field are also applied to the hyperpolarization device and the ¹³C isotope based contrast agent therein, such that the NV centers of the diamond material are polarized and the electron spins of the NV centres of the diamond material will then be transferred to ¹³C atoms when the Rabi frequency of the NV centres match the Larmor frequency of ¹³C.Referring to FIG. 2, there is shown a schematic representation of an embodiment of a hyperpolarization device 200 according to the present invention for enhancing polarization of 13C isotope-based magnetic resonance imaging contrast agents.

The device 200 comprises a diamond material structure 220 and one or more channels 205 provided adjacent to said diamond material structure. The diamond material structure 220 provides a source of negatively charged nitrogen vacancy (NV-) centres for polarization of a 13C isotope-based magnetic resonance imaging contrast agent disposed in said one or more channels. Furthermore, the diamond material structure 220 provides a light guide for light for excitation of nitrogen vacancy (NV-) centres for polarization of said 13C isotope-based magnetic resonance imaging contrast agent.

As is shown, the device 200 includes multi-hole channels 205 single-end opened cylindrical negatively charged nitrogen vacancy (NV-) enriched diamond tubes 210.

The hyperpolarization device 200 as shown in FIG. 2, in use, is placed in a resonator including an electron paramagnetic resonator (EPR) and a nuclear magnetic resonator (NMR) with an optical perturbation channel.

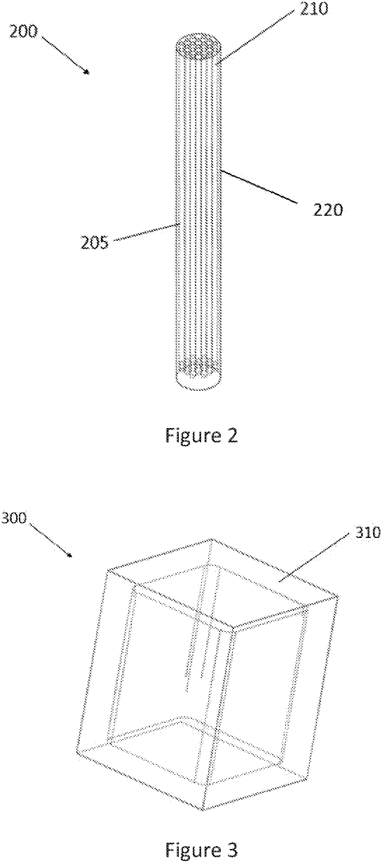

Referring now to FIG. 3, there is shown a schematic representation of an embodiment of a hyperpolarization casing 300 according to the present invention.

As is shown in FIG. 3, of the hyperpolarization casing 300 there is shown a diamagnetic outer cover, shell, or cage 310 of the instrument, which enables magnetic shielding from external magnetic source including the MRI machine.

In accordance with the present invention, the electron paramagnetic resonator (EPR) and a nuclear magnetic resonator (NMR) can be operated simultaneously or separately operated.

Both of the EPR system and NMR system can be operated in continuous wave (CW) mode and also in pulsed mode.

The CW mode of EPR means a static microwave and continuously tuneable static magnetic can be applied to the samples during operation. The pulsed mode of electron paramagnetic resonator (EPR) means a pulsed microwave and fixed static magnetic can be applied to the samples during operation.

Similarly, the CW mode of NMR means a static radio frequency (RF) wave and continuously tuneable static magnetic can be applied to the samples during operation, and the pulsed mode of electron paramagnetic resonator (EPR) means a pulsed RF wave and fixed static magnetic were applied to the samples during operation.

The optically perturbed channel is made of laser or LED with the wavelength in a range from 500 nm to 600 nm. The optically perturbed channel can be operated in CW mode or pulsed mode.

Referring specifically to FIG. 2, the embodiment of the hyperpolarization device 200 includes a diamond material structure 220 that is made of a multi-hole channels single-end opened cylindrical negatively charged nitrogen vacancy (NV-) enriched diamond tubes 210, which can store required amounts of samples of a ¹³C isotope-based Magnetic resonance imaging (MRI) contrast agents, for use in subsequent MRI imaging of a patient.

The device 200 has a transparent bottom, which enables the optical perturbation enters from the bottom side.

A diamagnetic metallic coating is made at the device’s 200 outer side surface, which will trap and scatter light between the inner space of the sample holder and the contained samples. Moreover, the device 200 is reusable after an independent and appropriate disinfection procedure.

A pump-injection channel is connected to the open-end of the device 200 during the operation in order to inject the hyperpolarized samples to the patient in a short period for the MRI imaging use in the patient. The samples used in the instrument are ¹³C isotope-based contrast agents. The samples for use as a contrasting agent, can be ¹³C enriched pyruvate, for example.

The hyperpolerisation device 200 can be made of synthetic diamonds, for example chemical vapor deposition (CVD) diamonds, CVD diamonds are synthesized by chemical vapor deposition, which allows carbon atoms in a gas to settle on a substrate in crystalline diamond form.

Using CVD, properties such as the shape of the synthetic diamond can be well controlled during its growth, such that a hyperpolarization device 200 in the shape of multi-hole channels single-end opened cylinders, which can be easily produced.

This is advantageous over the use of natural diamonds which requires accurate cutting from the raw diamond.

The hyperpolerisation device 200 also acts the source of NV- centers, which upon excitation, electron spins within the NV centres of the hyperpolerisation device 200. The electron spins of the NC centres can then be transferred to ¹³C atoms when the Rabi frequency of the NV centres match the Larmor frequency of ¹³C.

Within the present embodiment, the diamond material structure 220 is depicted as being formed from a single piece of diamond material, with columns defining the channels therebetween.

However, as should be appreciated and understood, the diamond material structure of the device may be comprised of more than one pieces of diamond material, and the device may also include other materials which may form part of a boundary of a channel. However, provided that the diamond material structure is arranged such that a channel is provided for the adjacent introduction of contrast agent, and the diamond material structure can act as a light guide for the hyperpolarisation as is herein described, such an embodiment will be understood to fall within the scope of the present invention,

As such, one or diamond material structures may be arranged to form a channel or part thereof adjacent the diamond material, or a channel may extend at least partly though a diamond material structure.

The present invention provides no structural limitations on the shape, geometry or size of the diamond material structure.

### Examples of the Invention

The system and device can be placed in same room with MRI machine in a clinical environment, or in a separated room.

The system may contain a samples holder placed in a resonator composited of an electron paramagnetic resonator and a nuclear magnetic resonator with an optical perturbation channel.

The instrument can have a diamagnetic outer cover, shell, or cage of the instrument, which enables magnetic shielding from external magnetic source including the MRI.

The electron paramagnetic resonator and the nuclear magnetic resonator can be operated simultaneously, or separately.

Both of the electron paramagnetic resonator and the nuclear magnetic resonator can be operated in continuous wave (CW) mode and also in pulsed mode.

The CW mode of electron paramagnetic resonator means a static microwave and continuously tuneable static magnetic can be applied to the samples during operation.

The pulsed mode of electron paramagnetic resonator means a pulsed microwave and fixed static magnetic can be applied to the samples during operation.

The CW mode of nuclear magnetic resonator means a static radio frequency (RF) wave and continuously tuneable static magnetic were applied to the samples during operation.

The pulsed mode of electron paramagnetic resonator means a pulsed RF wave and fixed static magnetic can be applied to the samples during operation.

The optically perturbed channel may be provided by laser or LED, or any appropriate light source.

The wavelength of the optically perturbed channel is in the range from 500 nm to 600 nm.

The optically perturbed channel can be operated in CW mode and in pulsed mode.

The samples holder may be a multi-hole channels single-end opened cylindrical diamond tubes, which can store required number of samples.

The device may be made of negatively charged nitrogen vacancy (NV-) enriched diamond.

The device may have a diamagnetic metallic coating at its outer side surface.

The diamagnetic metallic surface coating provides trapping and scattering of light between the inner space of the device and the contained samples of agent.

The device enables the optical perturbation enters from the bottom side of the samples holder.

The device may be reusable after disinfection.

A pump-injection channel may be connected to the open-end of the sample holder during the operation in order to inject the patient in short period for the MRI imaging use.

The samples are preferably ¹³C isotope-based contrast agents.

### Enhancing the Hyperpolarizing Effect of the ¹³C Isotope-Based Contrast Agent

In an embodiment of the present invention, the hyperpolarizing effect of the ¹³C isotope-based contrast agent can be enhanced by manipulating the nitrogen vacancy electron spin within the diamond with a plasmon-assisted method.

As is known by people within the art, surface plasmons (SPs) are coherent delocalized electron oscillations that exist at the interface between any two materials where the real part of the dielectric function changes sign across the interface (e.g. a metal-dielectric interface, such as a metal sheet in air).

In accordance with the present invention, surface plasmons are provided onto the surface of the diamond material of the hyperpolarisation device by illuminating a highly concentrated light beam to the surface of the diamond material, which is treated to be plasmonic-based or photonic based.

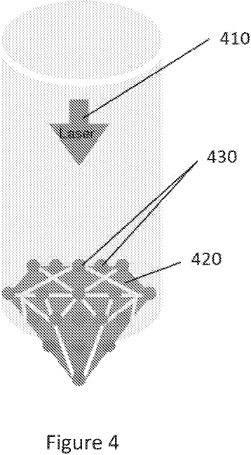

As is shown in FIG. 4, upon the illumination of a highly focused laser 410 onto the surface of a plasmonic-based or photonic based diamond 420, surface plasmons are formed 430 are formed on the surface of the diamond 420.

The treatment to the diamond material which causes it to be plasmonic-based or photonic based may be achieved by providing metallic coatings to the diamond surface or coupling the diamonds with metallic micro/nanostructures. Alternatively, the treatment may also refer to the forming of a dielectric coating to the diamond surface or coupling the diamonds with dielectric micro/nanostructures.

With the surface plasmons 430 present on the surface of the plasmonic-based or photonic based diamond material 420, the nitrogen vacancy electron spin of the diamond can be manipulated upon excitation by a light source, when subjected to an appropriate microwave field.

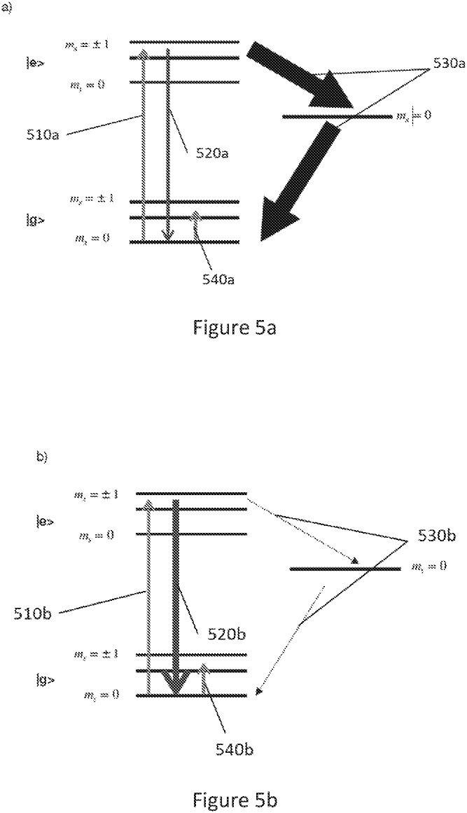

Referring now to FIGS. 5a and 5b which show the possible energy diagram of electron spin of the diamond material with or without the presence of surface plasmons on the surface of the plasmonic-based or photonic based diamond.

FIG. 5a shows an energy diagram of NV electron spin of diamond without surface plasmons. A microwave field 540a is provided to pump the nitrogen vacancy centers of the diamond to an electron spin active state. Upon excitation by a focused ion beam 510a, the excited nitrogen vacancy (NV-) centre will either return to the ground energy state by fluorescence decay 520a, or by emitting photons as indicated by arrows 530a.

In conventional hyperpolarisation methods where plasmons are not involved, the return of an excited nitrogen vacancy (NV-) centre to its ground energy level is usually predominant by the photons emission pathway 530a which has a lower electron spin active rate than that as provided by the fluorescence decay 520a. Thus, the overall hyperpolarisation rate of ¹³C isotope-based contrast agent will be hindered.

In contrast, with the presence of the surface plasmons, as is shown in the energy diagram of FIG. 5b, it has a higher opportunity for an excited nitrogen vacancy (NV-) centre to return to its ground energy level by fluorescence decay 520b rather than by the emission of photons 530b, upon the excitation by a focused light beam 510b and the presence of a microwave field 540b to pump the nitrogen vacancy center of the diamond to an electron spin active state.

Such a plasmonic-assisted method manipulates the nitrogen vacancy electron spin to predominantly undergo fluorescence decay which has a higher electron spin active rate, and thus leading to a more effective hyperpolarizing of ¹³C -samples.

### Disinfection System of the Hyperpolarisation Devices

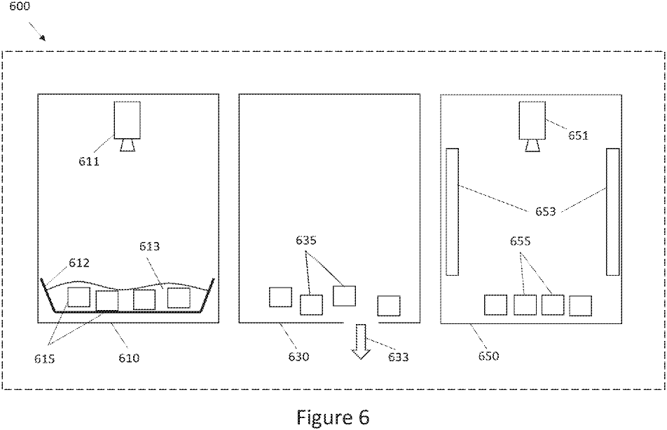

The hyperpolarisation devices of the present invention can be reused for multiple times upon appropriate cleaning and disinfection with the utilisation of a disinfection system as shown in FIG. 6.

FIG. 6 shows a disinfection system 600 for cleaning and disinfecting one or more hyperpolarisation device simultaneously. After providing activation to the ¹³C isotope-based MRI contrast agent, the hyperpolarisation devices are sent to the disinfection system 600 for thorough cleaning, which can then be reused after the disinfection process.

As is shown in FIG. 6, the disinfection system 400 includes three chambers, these being: (i) a disinfection chamber 610, (ii) a disposal chamber 630, and (iii) a drying chamber 650.

The disinfection chamber 610 provides the major cleaning and disinfection function to the one or more used hyperpolarisation devices 615. There is arranged a reservoir 612 which contains fluid 613 such as cleaning agent, organic fluid and water, the one or more used hyperpolarisation devices 615 are to be placed therein for thorough cleaning.

There is also arranged an ultraviolet light source 611 for emitting ultraviolet light to the used hyperpolarisation devices 615 and thereby disinfecting them. In an embodiment, the ultraviolet source can be provided by LED or laser, and the emitted ultraviolet light is in the wavelength ranging from 200 nm to 400 nm.

After cleaning and disinfection, the one or more hyperpolarisation devices 615 are then sent to the disposal chamber 630 wherein the disposal chamber 630 allows the disposal of any waste, including wastewater and retained cleansing agent, from the body of the hyperpolarisation devices 635. The waste is guided away from the disposal chamber 633 as is shown by the arrow 633.

In the last step of the disinfection process, the one or more hyperpolarisation devices 635 are sent to the drying chamber 650 wherein there is installed a drying unit 653 for drying the cleaned hyperpolarisation devices 655 and remove any liquid which are still retained thereon. The drying chamber 650 also includes an ultraviolet light source 651 which, same as that in the disinfection chamber, is used to further disinfect the hyperpolarisation devices 655 by irradiating ultraviolet light thereon.

In an embodiment, the ultraviolet light source may be a LED or a laser, and the wavelength of the ultraviolet light emitted ranges from 200 nm to 400 nm.

The drying chamber 650 provides sufficient room for multiple hyperpolarisation devices 655 to be dried simultaneously after the cleaning process, and therefore the cleaning and drying process can be carried out effectively and efficiently.

The present invention provides advantages, including enhanced hyperpolarisation time so as to be suitable for use in a contrasting agent in a clinical environment for MRI investigation, no additional elements need to be introduced in the contrasting agent, and direct delivery of the contrasting agent to a patient.

1. A device for enhancing polarization of ¹³C isotope-based magnetic resonance imaging contrast agents, said device comprising:
one or more diamond material structure and one or more channels provided adjacent to the diamond material structure;
wherein said diamond material structure provides a source of negatively charged nitrogen vacancy (NV-) centres for polarization of a ¹³C isotope-based magnetic resonance imaging contrast agent disposed in said one or more channels; and;
wherein said diamond material structure provides a light guide for light for excitation of nitrogen vacancy (NV-) centres for polarization of said ¹³C isotope-based magnetic resonance imaging contrast agent.

2. A device according to claim 1, wherein the device includes a plurality of plurality of columns defining said channels therebetween, wherein said columns are formed from said diamond material structure;.

3. A device according to claim 1 or claim 2, wherein said diamond material structure is formed from synthetic diamond.

4. A device according to claim 1 or claim 2, wherein said diamond material structure is formed from CVD (chemical vapor deposition crystal formation) diamond.

5. A device according to claim 1 or claim 2, wherein said diamond material structure is formed from HPHT (high-pressure high-temperature) diamond.

6. A device according to any one of the preceding claims, wherein said light is applied by an optical laser.

7. A device according to any one of the preceding claims, wherein said light is provided by a pulse laser.

8. A device according to any one of claims 1 to 6, wherein said light is provided by a continuous laser.

9. A device according to any one of the preceding claims, wherein the light is monochromatic.

10. A device according to any one of the preceding claims, wherein the ¹³C isotope-based magnetic resonance imaging contrast agents include pyruvate.

11. A system for enhancing polarization of ¹³C isotope-based magnetic resonance imaging contrast agents, said system comprising:
a device according to any one of claims 1 to 10;
a light source for optical pumping and providing excitation to the electron spins of the nitrogen vacancy (NV-) centres;
a radio frequency transmitter for providing a radio frequency signal;
a microwave transmitter for providing a microwave field, such that the NV centers are polarized and such that the electron spins of the NV centres are transferred to ¹³C atoms upon the Rabi frequency of the NV centres matching the Larmor frequency of ¹³C.
a tuneable electromagnet for providing a magnetic field, such that the nitrogen vacancy (NV-) centres electron spin active states of the nitrogen vacancy (NV-) centres are sufficiently separated, in order to provide unified electron spins states within the diamond under the presence of a microwave field.

12. A system according to claim 11, wherein the radio frequency signal is a static radio frequency wave.

13. A system according to claim 11, wherein the radio frequency signal is a pulsed signal.

14. A system according to claim 11, wherein the microwave is a continuous wave.

15. A system according to claim 11, wherein the microwave is a pulsed signal.

16. A system according to claim 11, wherein the electromagnet is a tuneable magnet.

17. A process for enhancing polarization of ¹³C isotope-based magnetic resonance imaging contrast agents for subsequent MRI imaging, said process including the steps of:
(i) providing a device according to any one of claims 1 to 10;
(ii) introducing a ¹³C isotope-based magnetic resonance imaging contrast agent with the channels of said device;
(iii) polarizing said ¹³C isotope-based magnetic resonance imaging contrast agent.

18. A system for cleaning and disinfecting the device for enhancing polarization of ¹³C isotope-based magnetic resonance imaging contrast agents as claimed in any one of claims 1 to 10, said system comprising:
(i) a disinfection chamber, for cleaning and disinfecting said one or more devices simultaneously;
(ii) a disposal chamber, wherein any waste retained on said one or more devices are disposed from the system, and
(iii) a drying chamber for drying the cleaned one or more said devices.

19. The system according to claim 18, wherein the disinfection chamber includes an ultraviolet light source for disinfection of said one or more devices.

20. The system according to claim 19, wherein said ultraviolet light source is in the wavelength of 200 nm to 400 nm.

21. The system according to any one of claims 18 to 20, wherein said ultraviolet light source is provided by LED or laser.

22. The system according to any one of claims 18 to 21, where said disinfection chamber has a reservoir for storing organic liquid, and any kinds of water, which allows said one or more devices to be cleaned therein.

23. The system according to any one of claims 18 to 22, wherein said drying chamber utilises furnace or electromagnetic wave as heat source for the drying purpose.

24. The system according to any one of claims 18 to 23 wherein the drying chamber includes an ultraviolet light source for disinfection of said one or more devices.

25. The system according to claim 24, wherein the ultraviolet light source is in the wavelength of 200 nm to 400 nm.

26. The system according to claim 24 or claim 25, wherein the ultraviolet light source is provided by LED or laser.

27. A process for enhancing the hyperpolarizing effect of the ¹³C -isotope based contrast agent provided by the device as claimed in any one of claims 1 to 10, said process including the steps of:
(i) treating said diamond material structure such that the diamond material is plamonic-based or photonic-based;
(ii) illuminating a light source to said diamond material structure for optical pumping and providing excitation to the electron spins of the nitrogen vacancy (NV-) centres, and as such to provide surface plasmons on the surface of said diamond material structure; and
(iii) providing electromagnetic perturbation to said device.

28. The process according to claim 27, wherein said diamond material is treated by forming a metallic coating on the surface of the diamond material structure.

29. The process according to claim 27, wherein said diamond material is treated by coupling the diamond material with metallic micro or nanostructures.

30. The process according to claim 27, wherein said diamond material is treated by forming a dieletric coating on the surface of the diamond material structure.

31. The process according to claim 27, wherein said diamond material is treated by coupling the diamond material structure with dieletric micro or nanostructures.

32. The process according to any one of claims 27 to 31, wherein the electromagnetic perturbation is provided by any one of microwave source, radio frequency wave source, or the like.
